(19) United States
(12) Patent Application Publication
(10) Pub. No.: US 2020/0390611 A1
(43) Pub. Date: Dec. 17, 2020

(54) Therapeutic Kinesiology Tape

(71) Applicant: Nuee Innovation, LLC, La Verne, CA (US)

(72) Inventor: Matthew Ryczek, Glendora, CA (US)

(21) Appl. No.: 16/773,507

(22) Filed: Jan. 27, 2020

Related U.S. Application Data
(60) Provisional application No. 62/861,468, filed on Jun. 14, 2019.

(51) Int. Cl. A61F 13/02; A61K 9/70; A61K 31/352; A61K 31/05

(52) U.S. Cl. CPC A61F 13/0246; A61F 13/0289; A61K 31/05; A61K 9/7038; A61K 31/352; A61F 13/0259

(57) ABSTRACT

A therapeutic kinesiology tape has an elastic fabric with a back side and a face side, the elastic fabric containing both lateral and longitudinal fibers which intersect to form a woven layer. The therapeutic kinesiology tape further has an adhesive material formulated with a transdermal cannabinoid formula and disposed on the back side of the elastic fabric, wherein a dose of the cannabinoid is transferred to the user after transdermal administration.

### CROSS-REFERENCE TO RELATED APPLICATIONS

This application for a utility patent claims the benefit of U.S. Provisional Application No. 62/861,468 filed 14 Jun. 2019.

### BACKGROUND OF THE INVENTION

### Field of the Invention

This invention relates generally to therapeutic kinesiology tape, and more particularly to a therapeutic kinesiology tape with a unique adhesive layer including cannabinoids.

### Description of Related Art

Rolf, U.S. Pat. No. 6,096,333, teaches an adhesive patch that includes medications integrated into the adhesives of the patch. Weimann, U.S. Pat. No. 9,962,340, teaches a similar patch.

Wust, U.S. 2012/0232448, teaches a kinesiology adhesive tape having an elastic woven fabric and an adhesive coating layer that includes spherical fascial bodies that ay be given an alkaline pH value via an alkaline solution.

Longo, U.S. 2016/0220440, teaches a kinesiology adhesive tape that includes a woven fiber layer, an adhesive layer engaged with the first side of the woven fiber layer, and a protective layer engaged with the first side of the adhesive layer.

### SUMMARY OF THE INVENTION

The present invention teaches certain benefits in construction and use which give rise to the objectives described below.

The present invention provides a therapeutic kinesiology tape comprising an elastic fabric having a back side and a face side, the elastic fabric containing both lateral and longitudinal fibers which intersect to form a woven layer. The therapeutic kinesiology tape further includes an adhesive material formulated with a transdermal cannabinoid formula and disposed on the back side of the elastic fabric, wherein a dose of the cannabinoid is transferred to the user after transdermal administration.

A primary objective of the present invention is to provide a therapeutic kinesiology tape having advantages not taught by the prior art.

Another objective is to provide a therapeutic kinesiology tape which has a woven layer with a transdermal cannabinoid formula disposed thereupon.

A further objective is to provide a therapeutic kinesiology tape which provides a unique combination of therapeutic uses, such as simultaneous relief from muscle pain and other ailments that may be treated using a cannabinoid or therapeutic tape.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is a perspective view of a section of kinesiology tape according to one embodiment of the present invention;

FIG. 2 is a front plan view of a person wearing the kinesiology tape of FIG. 1;

FIG. 3 is an up-close view of a first layer of the kinesiology tape; and

FIG. 4 is a side sectional view of the kinesiology tape.

### DETAILED DESCRIPTION OF THE INVENTION

The above-described drawing figures illustrate the invention, a kinesiology tape having a unique adhesive layer whose formulation provides therapeutic benefits. The invention further includes a method of use in which the kinesiology tape may be applied during therapy or recreation to reduce soreness in overused and injured muscles and in rehabilitation to accelerate recovery.

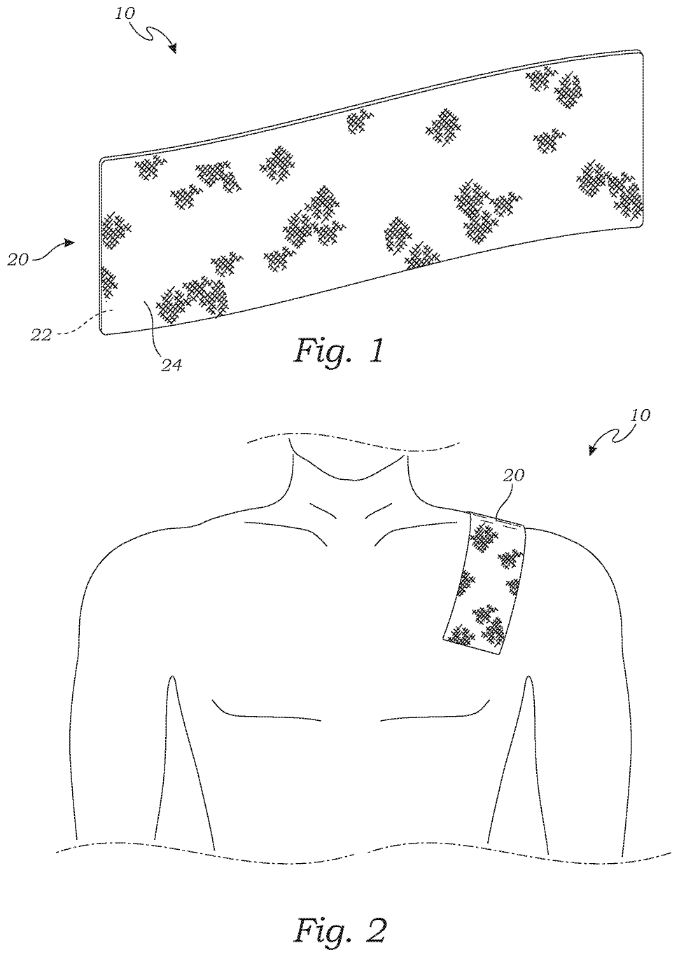

FIG. 1 is a perspective view of kinesiology tape 10 according to one embodiment of the present invention. FIG. 2 is a front plan view of a person wearing the kinesiology tape 10 shown in FIG. 1. The kinesiology tape 10 (a.k.a. therapeutic elastic tape, or KT tape), includes an elastic fabric 20 having a back side 22 and a face side 24. The back side 22 includes an adhesive material 30, and may include a release liner 32, both shown in FIG. 4, for removably attaching the tape 10 to the user. As shown in FIGS. 1-2, the tape 10 may be in the form of a generally rectangular strip, which may be provided in the form of a roll or a pre-cut strip. However, other embodiments of the tape 10 may have different shapes and configurations for different placements on persons, and for use by persons of different sizes.

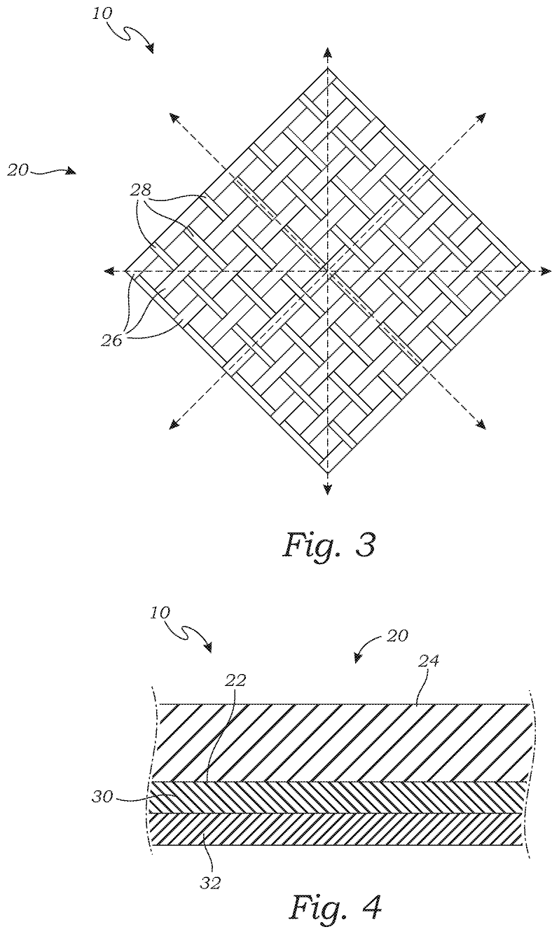

FIG. 3 is an up-close view of the elastic fabric 20. As shown in FIG. 3, the elastic fabric 20 includes both lateral and longitudinal fibers 26 & 28 which intersect to form a woven layer. The fibers 26 and 28 are elastic, and provide suitable stretching characteristics in both lateral and longitudinal directions, for optimum performance of the tape 10. In some embodiments, the elastic fabric 20 is formed by a combination of elastic and non-elastic fibers. In some embodiments, the fibers 26 & 28 include both elastic synthetic materials, and also cotton material, but in other embodiments may be covered in or woven with other organic or synthetic materials. This construction is described in greater detail in Longo, U.S. 2016/0220440, which is hereby incorporated by reference in full.

FIG. 4 is a side sectional view of the KT tape 10, illustrating the elastic fabric 20, the adhesive material 30, and the release liner 32 mentioned above. In this embodiment, the adhesive material 30 may be rolled onto the elastic fabric 20 through a laminating process, though the adhesive 30 may be applied though any other means known to those skilled in the art. The adhesive material 30 may further be combined with other ingredients intended for therapeutic purposes, or other ingredients which assist with adhesion to the skin, moisturizing, transfer agents, fragrances, etc., described in detail herein.

In this embodiment, the adhesive material 30 is combined with a cannabinoid, and/or other ingredients for diffusion to a portion of a body of the user, examples of which are provided below. The cannabinoid formula is configured for transdermal delivery and may be deposited on portions of the face side 24 and/or back side 22 of the elastic fabric 20, such that the cannabinoid is delivered after application of the tape 10 to a user, described in greater detail below.

The present application also includes methods for treating a patient suffering from a condition such as pain, inflammation, nausea, convulsions, muscle spasms, depression, and cachexia comprising administering tape 10, as illustrated in FIG. 2 and described below. The specific formulation of the adhesive material 30 may be varied depending upon the condition(s) being treated, as can the location of application of the tape 10.

The tape 10 may further provide a lifting effect on the skin which can reduce swelling and inflammation by improving circulation and reduce pain by taking pressure off pain receptors. Taping patterns may constrain injured areas of a user to maintain proper functional movements. Additionally, different amounts of stretch can be applied to the tape 10 prior placement, to be different amounts of force on or around the muscle and injury. Multiple pre-cut straps may be used at a time to effect counter-force and/or additional support, or the tape 10 may be cut to conform to specific parts of the body. The tape 10 may be designed to be comfortable for long term wear up to 24 hours and may be usable in aqueous environments.

The cannabinoid should be formulated with the adhesive 30 so that a desirable dose is transferred to the user after transdermal administration.

In some embodiments, methyl salicylate may be included to provide a fragrance element, or in a high concentration as a rubefacient and analgesic to treat joint and muscular pain. However, in the body it metabolizes into salicylates, including salicylic acid, a known NSAID. Some embodiments may include menthol, for its local anesthetic and counterirritant qualities, alone or combined with chemicals such as camphor or capsaicin (as a topical analgesic). Minute amounts of camphor are readily absorbed through the skin, where it stimulates nerve endings sensitive to heat and cold, producing a warm sensation when vigorously applied, or a cool sensation when applied gently. Camphor may also provide a fragrance element.

Other anti-inflammatories and/or analgesics may include Arnica Montana flower extract, Calendula Officinallis extract (which may also have anti-viral and/or anti-genotoxic effects), and Boswellia Carteril Resin Extract (which also may prevent the loss of cartilage). Arctium Lappa Root (Burdock) Extract may further be included as a diuretic, diaphoretic, and/or a blood purifying agent.

Aloe Barbadensis Leaf Extract, commonly known as Aloe Vera, may be added as a moisturizer and anti-irritant. In some embodiments, Camellia is added to provide a nutritive effect. Benzoin resin may be added for additional fragrance, while Carbomer may assist in providing a gel structure. While suggested ingredients are provided above, other ingredients may be included for therapeutic, recreational, or structural purposes, in as much as the ingredients are known in the art.

In some embodiments, the cannabinoid is a cannabinol, such as THC or CBN, or in other embodiments, the cannabinoid may be a cannabidiol, such as CBD. The cannabinoid may be formulated using a cannabinoid acid comprising a carboxylic acid substituent attached to an aromatic ring such as THCa or CBDa. In some embodiments, the cannabinoid is HTCv or CBC. Mixtures of two or more cannabinoids may also be used; for example, CBD and THC may be used in a 1:1 ratio.

The clinical usefulness of the cannabinoids, including tetrahydrocannabinol (THC), to provide analgesia, help alleviate nausea and emesis, as well as stimulate appetite has been well-recognized. Cannabinoids offer a variety of pharmacological benefits, including, but not limited to, anti-spasmodic, anti-inflammatory, anti-convulsant, anti-psychotic, anti-oxidant, neuroprotective, anti-cancer, and immunomodulatory effects.

Therefore, the combination of the kinesiology tape 10 and the transdermal cannabinoid and/or other therapeutic ingredients provides a method for uniquely relieving muscle pain and other ailments that may be treated using a cannabinoid or therapeutic tape.

As used in this application, the words “a,” “an,” and “one” are defined to include one or more of the referenced item unless specifically stated otherwise. The terms “approximately” and “about” are defined to mean +/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

What is claimed is:

1. A therapeutic kinesiology tape comprising:
an elastic fabric having a back side and a face side, the elastic fabric containing both lateral and longitudinal fibers which intersect to form a woven layer; and
an adhesive material formulated with a transdermal cannabinoid formula to disposed on the back side of the elastic fabric, wherein a dose of the cannabinoid is transferred to the user after transdermal administration.

2. The therapeutic kinesiology tape of claim 1, wherein the elastic fabric is formed by a combination of elastic and non-elastic fibers.

3. The therapeutic kinesiology tape of claim 1, wherein the adhesive material is rolled onto the elastic fabric through a laminating process.

4. The therapeutic kinesiology tape of claim 1, wherein a release liner is mounted on the adhesive material, and wherein the release liner is removed prior to application of the therapeutic kinesiology tape on the user for transdermal administration of the cannabinoid formula.

5. The therapeutic kinesiology tape of claim 1, wherein the therapeutic kinesiology tape is pre-cut to conform to a body part of the user.

6. The therapeutic kinesiology tape of claim 1, wherein the transdermal cannabinoid formula is a cannabinol.

7. The therapeutic kinesiology tape of claim 1, wherein the transdermal cannabinoid formula is a cannabidiol.

8. The therapeutic kinesiology tape of claim 1, wherein the transdermal cannabinoid formula is formulated using a cannabinoid acid comprising a carboxylic acid substituent attached to an aromatic ring.

9. The therapeutic kinesiology tape of claim 1, wherein the transdermal cannabinoid formula comprises a mixture of at least two cannabinoids.

10. The therapeutic kinesiology tape of claim 9, wherein the two cannabinoids are CBD and THC used in a 1:1 ratio.

11. A method of using a therapeutic kinesiology tape, the method comprising the steps of:
providing at least one therapeutic kinesiology tape comprising an elastic fabric having a back side and a face side, the elastic fabric containing both lateral and longitudinal fibers which intersect to form a woven layer;
providing an adhesive material disposed on the back side of the elastic fabric such that the therapeutic kinesiology tape may be taped to a body part of a user;
taping the back side of at least one therapeutic kinesiology tape onto the body part of the user, and stretching at least one therapeutic kinesiology tape such that the body part no longer has full range of motion;
to providing a transdermal cannabinoid formulated with the adhesive material such that a dose of the cannabinoid formula is transferred to the user after at least one therapeutic kinesiology tape is taped onto the body part of the user.

12. A method of using a therapeutic kinesiology tape, the method comprising the steps of:
providing at least one therapeutic kinesiology tape comprising an elastic fabric having a back side and a face side, the elastic fabric containing both lateral and longitudinal fibers which intersect to form a woven layer;
providing an adhesive material disposed on the back side of the elastic fabric such that the therapeutic kinesiology tape may be taped to a body part of a user;
providing a release liner mounted on the adhesive material;
removing the release liner such that the adhesive material is exposed;
taping the back side of at east one therapeutic kinesiology tape onto the body part of the user, and stretching at least one therapeutic kinesiology tape such that the body part no longer has full range of motion;
providing a transdermal cannabinoid formulated with the adhesive material such that a dose of the cannabinoid formula is transferred to the user after at least one therapeutic kinesiology tape is taped onto the body part of the user.
